(19) United States
(12) Patent Application Publication
(10) Pub. No.: US 2025/0195410 A1
(43) Pub. Date: Jun. 19, 2025

(54) TOPICAL PHARMACEUTICAL COMPOSITION WITH ANTI-INFLAMMATORY AND ANALGESIC ACTIVITY AND USES THEREOF

(71) Applicant: GLYCORES 2000 SRL, Milano MI (IT)

(72) Inventors: Giorgio Zoppetti, Milano (IT); Pasqua Anna ORESTE, Milano (IT)

(73) Assignee: GLYCORES 2000 SRL, Milano MI (IT)

(21) Appl. No.: 18/849,545

(22) PCT Filed: Mar. 25, 2022
(86) PCT No.: PCT/IB2022/052773

§ 371 Date: Sep. 23, 2024

(51) Int. Cl. A61K 9/00; A61K 9/08; A61K 31/196; A61K 47/10; A61K 47/14; A61K 47/34

(52) U.S. Cl. CPC A61K 9/0014; A61K 9/08; A61K 31/196; A61K 47/10; A61K 47/14; A61K 47/34

(57) ABSTRACT

The present invention relates to a topical pharmaceutical composition having anti-inflammatory and analgesic activity to be delivered preferably by a vaporisation (spraying) system, comprising diclofenac or a pharmaceutically acceptable salt thereof, volatile silicones having low dynamic viscosity and high molecular weight silicon compounds. The invention also relates to the use of said composition especially in the treatment of pain and inflammation.

### TECHNICAL BACKGROUND

Diclofenac, [(2,6-dichlorophenyl)amino]benzeneacetic acid, initially developed as sodium salt for solid oral or injectable formulations, has also been considered for topical applications as creams, gels or dressings and patches. The formulation of diclofenac sodium in creams, gels or plaster is very problematic due to its low solubility in water. For the same reason, injectable formulations of diclofenac sodium usually contain a mixture of water and solvents, like in Voltaren®, wherein diclofenac sodium is solubilized in a mixture of water and propylene glycol.

Several lipophilic mixtures of diclofenac sodium or potassium salt have been disclosed (WO2004030665. WO9857624, EP0834312), and also liposomal systems were developed (US2004121987). A gel and a foam containing diclofenac sodium have also been described, said formulations containing dimethylsulfoxide (WO2008049020, WO2011112875).

Further attempts to deliver diclofenac sodium for topical application were described but, notwithstanding diclofenac sodium low dosage, turbid water-based gels were obtained (WO2004017998). Moreover, also diclofenac sodium volatile-based gels were disclosed (WO2009007764). Further, diclofenac acid was ion paired with biologically active molecules to obtain a water solution (WO2010087947).

In view of the above, it is evident that in order to develop a bioavailable, stable, topical formulation comprising diclofenac, especially diclofenac sodium, hydrophobic solvents or lipophilic solubilizing agents have to be used.

Considering that, in order to obtain an effective skin permeation, the active ingredient shall have a favourable water/oil partition ratio, and that diclofenac alkali salts are oriented toward the lipophilic fraction, as an alternative to sodium or potassium salts, salts with organic amines have been studied, such as ethylamine, diethylamine, 1-(2-hydroxyethyl)pyrrolidine (also known as 2-pyrrolidinemethanol), 1-(2-hydroxyethyl)pyrrolidine and epolamine. Indeed, the study of the solubility in water and in solvent described in the paper of Minghetti et al., “Ex vivo study of transdermal permeation of four diclofenac salts from different vehicles”, J. Pharm. Sci, 96 n. 4 pp. 814-914 (2007) confirms that diclofenac organic salts are more soluble in water, while sodium and potassium salts are more soluble in lipophilic solvents. In particular, diclofenac epolamine salt is very soluble in water and comparatively less soluble in organic solvents. This high water solubility of diclofenac epolamine has been described also by Fini et. al. “Diclofenac/N-(2-hydroxyethyl) pyrrolidine: a new salt for an old drug.” Drugs Exptl. Clin. Res., 1993, XIX: 81-88, wherein the water solubility of diclofenac epolamine is reported to be 40% p/v.

Pharmaceutical formulations containing diclofenac salts with organic bases, like diethylamine or epolamine, were described in form of gels or water-based solutions (EP2055298, CN101224186, WO2010060798). Diclofenac sodium and epolamine salts have been also described in the preparation of topical semisolid patches (WO2011049058, EP0621263), and in the case of patches comprising diclofenac epolamine, pure water is used to make a concentrate solution of the active ingredient.

All the formulations above described in gel, patch or solution show different application problems. For instance, creams with sodium or potassium salts show low skin permeation, which can be enhanced for example formulating gels in presence of dimethylsulfoxide, being however aware that dimethylsulfoxide has noxious side effects. Ethylenamine salt seems to have good permeation properties, but the use of substances producing nitrosamines has been recently discouraged by Heath Authorities. Finally, the epolamine salt is useful for the preparation of water-based patches or gels, but the skin permeation of the formulations thus obtained is still low, due to the weak capability of the semisolid gel to release the diclofenac molecule.

### PRIOR ART

In CN1150541 a spray formulation containing diclofenac sodium and various solubilizers such as Tween 80, propylene glycol and glycerin is described. In said formulation, diclofenac (10 and 20 gr) and the solubilizers are dissolved in water, thus confirming that, in order to prepare a diclofenac sodium a water-based composition for external use, like gel or spray, solubilizing agents are essential.

WO2009/047785 discloses a composition in a non-water-based solution for topical use. Said composition includes an effective amount of a pharmaceutical acceptable diclofenac salt, in particular the sodium salt or diethylamine salt, solubilized in a short-chain alcohol in a percent from 10 to 39 v/v, in presence of a skin permeation enhancer, a solvent consisting of propylene glycol, glycofurol or of a mixture thereof, and optionally of a humectant or antioxidant agent and of another agent favouring the permeation, and in which the short-chain alcohol is C₂-C₅, in particular ethanol in an amount of about 10-20% v/v. The solvents described in this document, glycofurol and propylene glycol, are high boiling point solvents.

In Italian patent n. 1413980, diclofenac or a salt thereof with an organic base, in particular with a cyclic organic base selected from hydroxyethylpyrrolidine and hydroxyethylpiperidine, is mixed to a volatile silicon, and a skin permeation enhancer agent. The formulation thus obtained showed to be much more topically bioavailable than a commercial topical gel composition comprising diclofenac epolamine.

There is an existing need to provide new topical compositions comprising diclofenac which overcome the above mentioned drawbacks of the prior art.

### OBJECTS OF THE INVENTION

It is an object of the invention to provide a new composition comprising diclofenac or a pharmaceutically acceptable salt thereof, which is topically highly bioavailable, i.e. which shows an improved permeation through the skin.

It is a further object of the invention to provide a now topical composition comprising diclofenac or a pharmaceutically acceptable salt thereof, which is stable and easy to administer.

It is another object of the invention to provide the use of the new composition of the invention in the topical treatment of inflammation and pain.

### BRIEF DESCRIPTION OF THE DRAWINGS

FIG. 1 shows a schematic representation of a Franz's type diffusion cell.

FIG. 2 shows the permeation curves of the compositions of Examples 1-3 compared with a reference compound (Comparative Example 1).

FIG. 3 shows the role of the amount of the high molecular weight silicon compound in the composition in the permeation, in particular the permeation curves of the compositions of Examples 1-3 compared with a reference compound (Comparative Example 1) at 1, 8 and 24 hours (high molecular weight silicon compound: 0%=Comparative Example 1, 5%=Example 1, 4%=Example 2 and 6%=Example 3).

### DETAILED DESCRIPTION OF THE INVENTION

According to one of its aspects, a subject-matter of the present invention is a topical pharmaceutical composition comprising:
(a) diclofenac or one of its pharmaceutically acceptable salts;
(b) at least one volatile silicon compound having a low dynamic viscosity;
(c) at least one high molecular weight silicon compound having a high dynamic viscosity;
(d) a solvent consisting of at least one saturated aliphatic alcohol having 2 to 4 carbon atoms, preferably a mixture of two saturated aliphatic alcohols having 2 to 4 carbon atoms;
(e) at least one permeation enhancer; and
(f) optionally a refreshing agent.

According a preferred embodiment, the topical pharmaceutical composition of the invention comprises:
(a) 0.1 to 3.5%, preferably 0.5 to 3.0% by weight (with respect to the total weight of the composition) of diclofenac or one of its pharmaceutically acceptable salts;
(b) 30 to 60%, preferably 35 to 55% by weight (with respect to the total weight of the composition) of a volatile silicon having a low dynamic viscosity
(c) 3 to 6%, preferably higher than 3% to less than 6%, more preferably 4% to less than 6% by weight (with respect to the total weight of the composition) of one high molecular weight silicon compound having a high dynamic viscosity;
(d) 20% to 50% by weight (with respect to the total weight of the composition) of a solvent consisting of a mixture at least one saturated aliphatic alcohol having 2 to 4 carbon atoms, preferably a mixture of two saturated aliphatic alcohols having 2 to 4 carbon atoms;
(e) 2% to 15%, preferably 6% to 15% by weight (with respect to the total weight of the composition) of at least one permeation enhancer; and
(f) 0 to 3%, preferably 0.1% to 3% in weight (with respect to the total weight of the composition) of a refreshing agent.

According to the present invention, the term “topical composition” here indicates a locoregional composition which has to be applied to a localised area of the body or to one surface of a body in order to achieve a local effect and substantially does exert a systemic activity.

Diclofenac is the International Non-proprietary Name of [(2,6-dichlorophenyl)amino]benzeneacetic acid and is the active ingredient of the composition of the invention. When diclofenac weight % and dosages are herein given, they relate to diclofenac sodium equivalent amounts, regardless of the diclofenac derivative which is used, i.e. regardless the fact that diclofenac free acid or a different diclofenac salt is used.

According to the present invention, “pharmaceutically acceptable salts” are any salt which are non-toxic and physiologically compatible. According to a preferred embodiment, the pharmaceutically acceptable salts are selected from salts with an pharmaceutically acceptable organic base, preferably with a pharmaceutically acceptable amine, such as, but not limited to, methylamine, triethylamine, pyrrolidine, piperidine, morpholine, l-ethylpiperidine, 2-aminoethanol, dimethylaminoethanol, diethylaminoethanol (described in U.S. Pat. No. 3,558,690), pyrrolidinemethanol (epolamine) and piperidineethanol obtainable as described in EP 0271709A, more preferably pyrrolidineethanol (epolamine) and piperidineethanol.

According to the present invention, a “low dynamic viscosity” indicates a dynamic viscosity lower than or equal to 5 cP, preferably from 0.5 to 5 cP.

According to the present invention, a “high dynamic viscosity” indicates a dynamic viscosity higher than or equal to 40 cP, preferably from 40 to 400 cP.

According to the present invention, “volatile silicon compound having a low dynamic viscosity” indicates a (per)methylated polysiloxane having from 2 to 6 silicon atoms having a dynamic viscosity lower than 5 cP, preferably from 0.5 to 5 cP, for instance selected from C2-C6-polysiloxanes in which the silicon atoms are all methylated, such as hexamethyldisiloxane (for example “Liveo™ Q7-9180 Silicone Fluid 0.65 cSt”, with a dynamic viscosity of 0.59-0.71 cP), octamethyltrisiloxane (for example “Liveo™ Q7-9180 Silicone Fluid 1 cSt” with a dynamic viscosity from 0.9 to 1.1 cP) and decamethylpentasiloxane (for example “Livco™ ST-Cyclomethicone 5-NF” with a dynamic viscosity from 3.8 to 4 cP), said compounds being also marketed by other companies.

According to the present invention, “high molecular weight silicon compound having a high dynamic viscosity” indicates a high molecular weight silicon compound having a dynamic viscosity higher than 40 cP, preferably from 40 to 400 cP, for example about 40 cP, preferably selected from polydimethylsiloxanes, such as the hydroxyl-terminated polydimethylsiloxane presently marketed as Dimethiconol 40 (Liveo® ST-Dimethiconol 40, Liveo® 360 Medical Fluid 20 cSt, Liveo® 360 Medical Fluid 100 cSt).

According to the present invention, the dynamic viscosity is measured at 25° C.

According to the present invention, the “solvent” consists of at least one saturated aliphatic alcohols having 2 to 4 carbon atoms, but preferably is a mixture of two saturated aliphatic alcohols having 2 to 4 carbon atoms; said alcohols being preferably selected from ethanol, n-propanol, iso-propanol, n-butanol, iso-butanol, sec-butanol and tert-butanol. According to a preferred embodiment, said mixture is made of saturated aliphatic alcohols having 2 and 3 carbon atoms, preferably of only two alcohols, more preferably a mixture of ethanol and isopropanol, advantageously in a weight ratio from 1/0.4 to 1/3 respectively. According to the present invention, a “skin permeation enhancer” is a compound which favours the skin permeation of the composition, allowing the improved permeation of the active ingredient through the skin. Several skin permeation enhancers are known to the skilled in the art. According to a preferred embodiment, the skin permeation enhancer is selected from fatty alcohols, such as lauric alcohol; or fatty acids, such as linolenic acid or oleic acid; fatty acid esters, such as isopropyl palmitic ester, isopropyl stearic ester, isopropyl linoleic ester, isopropyl oleate and isopropyl myristate; glycerol or saccharose monostearate, glycerol or saccharose monolinoleate and glycerol or saccharose monooleate; and fatty alcohol ethers from 10 to 20 carbon atoms. Examples of skin permeation enhancers can also be found in Pharmaceutical Technology, November 1997, pp. 58-66.

According to a preferred embodiment, the component (a) is a diclofenac salt of an organic amine, and it is preferably diclofenac epolamine salt or diclofenac 1-(2-hydroxyethyl)piperidine salt.

According to a preferred embodiment the component (b) is a volatile silicon having a dynamic viscosity lower than 5, preferably from 0.6 to 4 cP, selected from the group consisting of hexamethyldisiloxane, octamethyltrisiloxane and decamethylcyclopentasiloxane.

According to a preferred embodiment the component (c) is a high molecular weight silicon compound, preferably Dimethiconol 40 as above defined.

According to a preferred embodiment, component (d) is an ethanoVisopropanol mixture, more preferably with a ratio from 1/0.4 to 1/3 w/w.

According to a preferred embodiment, the component (c) is isopropyl myristate.

According a preferred embodiment, component (f), when present, is menthol or camphor or a mixture of the two.

According a preferred embodiment, the topical pharmaceutical composition of the invention comprises:
(a) 0.1 to 3.5%, preferably 0.5 to 3.0% by weight (with respect to the total weight of the composition) of diclofenac or diclofenac epolamine salt or 1-(2-hydroxyethyl)piperidine salt;
(b) 30 to 60%, preferably 35 to 55% by weight (with respect to the total weight of the composition) of a volatile silicon selected from hexamethyldisiloxane, octamethyltrisiloxane and decamethylcyclopentasiloxane
(c) 3 to 6%, preferably higher than 3% to less than 6%, more preferably 4% to less than 6% by weight (with respect to the total weight of the composition) of Dimethiconol 40;
(d) 20% to 50% by weight (with respect to the total weight of the composition) of a solvent consisting of a mixture of ethanol/isopropanol in a volume ratio of 1/0.4 to 1/3;
(e) 2% to 15%, preferably 6% to 15% by weight (with respect to the total weight of the composition) of isopropyl myristate; and
(f) 0 to 3%, preferably 0.1% to 3% in weight (with respect to the total weight of the composition) of menthol or camphor, or a mixture of the two.

According to another of its aspects, subject-matter of the present invention is a multi-dose composition, in a 10 ml to 200 ml spray packaging, comprising:
0.100 to 7 g of diclofenac sodium or 0,130 to 9-g of diclofenac epolamine salt or 0,134 to 9,359 g of 1-(2-hydroxyethyl)piperidine salt;
3 to 120 g of a volatile silicon selected from hexamethyldisiloxane, octamethyltrisiloxane and decamethylcyclopentasiloxane
0.3 to 12 g of Dimethiconol 40;
2 to 100 g of a mixture of ethanol/isopropanol in a volume ratio of 1/0.4 to 1/3;
0.2 to 60 g of isopropyl myristate; and
0.0 to 6 g of menthol or camphor, or a mixture of the two.

The above multi-dose composition is contained in a spray device and provides for the administration of topical single doses of the composition.

The composition of the invention may be formulated according to any suitable method.

According to another of its aspects, subject-matter of the present invention is a process for the preparation of the composition of the invention which comprises:
i. adding, in any order the volatile silicon compound, the high molecular weight silicon compound, the skin permeation enhancer and the refreshing agent, if present, to a mixture of saturated aliphatic alcohols having 2 to 4 carbon atoms, under stirring at a temperature from 15 to 25° C., thus obtaining a solution; and
ii. subsequently adding diclofenac or a pharmaceutically acceptable salt thereof to the solution obtained in step (i), under stirring at a temperature from 15 to 25° C.

The preferred embodiments above disclosed, such as amounts of the components, ratios, etc., are also preferred embodiments of the process of the invention.

According to a preferred embodiment, in the process of the invention a weight of the ethanol/isopropanol mixture in a weight ratio from 1/0.4 to 1/3 (herein also referred to as alcoholic mixture) is prepared under mild stirring and, to this mixture, from 1.1 to 2.5 volumes of the silicon mixture with respect to the volume of the alcohol mixture, from 1 to 3 weight of isopropyl myristate, with respect to the weight of the alcohol mixture are added, in any order, keeping a mild stirring and the temperature from 15 to 25° C., thus obtaining a solution. Under the same conditions of stirring and temperature, menthol and/or camphor are added in an amount from 0.5 to 3% by weight with respect to the total weight of the above solution. Finally, diclofenac or a pharmaceutically acceptable salt thereof, preferably diclofenac epolamine or diclofenac piperidinethanol, is added in an amount from 0.1 to 5% by weight, preferably from 0.1 to 3% by weight (weight of diclofenac sodium equivalent) with respect to the total weight of the solution also comprising menthol and/or camphor.

The final composition above has a density (p/v) from 0.6 to 1.0 g/mL, preferably from 0.7 to 0.9 g/mL, more preferably from 0.75 to 0.85 g/mL, at a temperature of 25° C.

If needed or desired, the composition may also comprise further active ingredients or further pharmaceutically acceptable excipients.

The composition of the invention is in the form of a solution which is easily vaporizable, therefore suitable as a pharmaceutical composition for external use, especially useful in the treatment of pain and inflammation, such as muscular or arthritis pain, arthritic inflammation, muscular contractures and tendinitis.

The composition of the present invention showed improved skin permeation properties which can be evaluated with known analytical methods, for example with the model described by Shah V. P. et al in “Evaluation of test system used for in vitro release of drugs for topical dermatological drug products. Pharm. Dev. and Technol. 4(3), 377-385 (1999)”. In this test, a shaved biological membrane is mounted between two chambers in a two chamber cell (Franz cell), in which the receiving compartment is an aqueous buffer solution and represents the under skin blood stream, and the donor compartment represents the external skin area. Once the sample has been deposited in the donor compartment, the active principle released by the pharmaceutical compound is diffused through the different skin layers and then released in the receiving compartment. At specific timeframe, usually within 24 hours measured buffer samples are withdrawn from the receiving compartment and the active content is assayed.

With the data obtained a kinetic curve is constructed and the diffusion efficiency is calculated as the amount of active principle/used area (usually 1 cm²)/observation time (usually 24 hours) and expressed as μg/cm². At the end of the experiment the tissue used is minced in small pieces and the active extracted according to validated methodologies. The value obtained from the minced tissue represents the amount of active released from the pharmaceutical preparation fraction not diffused in the receiving compartment. The two values correspond to the total active released within the observation time frame (24 h). The results of the test carried out with representative compositions of the invention and with a comparative composition are discussed in the Experimental Section and shown in the drawings.

The composition of the invention for use in human and veterinary therapy, especially in the treatment of pain, inflammation, muscular or arthritis pain, muscular contractures and tendinitis, is another subject-matter of the invention.

According to another of its aspects, subject-matter of the invention is a method for treating inflammations such as muscular or arthritis pain, arthritic inflammation, muscular contractures and tendinitis, said method comprising topically administering an effective amount of the composition of the invention to a subject in need thereof. Said subject is preferably a mammal, including human beings, but also including pets, livestock and wild animals.

With respect to the composition disclosed in the Italian patent n. 141398, the composition of the invention shows an unexpected improved permeation, as it will be demonstrated in the Examples and in the Drawings enclosed.

Indeed, Applicant originally had hypothesised that the addition of increased amounts of a high molecular weight silicone compound was expected to prevent the permeation of the active ingredient (diclofenac) to some extent, but at the same time to allow the active ingredient stay longer on the skin, so that prolonging the permeation along time.

Surprisingly, and contrary to the expectations, the addition of a higher amount of the high molecular weight silicon compound, for instance in an amount higher than 3% and up to less than 6% by weight, with respect to the total weight of the composition, resulted in an increase of the permeation of the active ingredient with respect to the permeation provided by the composition disclosed in the Italian patent n. 141398 with no high molecular weight silicon compound. Further the addition of 6% of the high molecular weight silicon compound resulted in a relative lower permeation of the active, indicating that the optimal range to achieve the better permeation rate of the active of the high molecular weight silicone compound is from 4% to less than 6% in weight with respect to the total weight of the composition.

The permeation was evaluated by means of the pig membrane test and all the compositions containing from 4% to 6% by weight of the high molecular weight silicon compound, with respect to the total weight of the composition, show better permeations than the formulation with no high molecular weight silicon compound. In particular during all the time of observation, the composition containing 4% or 5% of high molecular weight silicon compound showed the maximum increase, while the formulations containing 6% of high molecular weight silicon compound show lower permeation rate, but nevertheless greater than the composition of the Italian patent above. The maximum permeation rates were obtained with 5% of high molecular weight silicon compound.

So, the composition of the present invention is a solution comprising diclofenac as the active ingredient, preferably in the form of the salts above defined, solubilized in a solvent mixture without water, in the presence of specific amounts of high molecular weight silicon compound, which showed to be particularly advantageous for skin permeation in comparison of the formulation containing no high molecular weight silicon compound.

The composition of the invention may be packaged in single- or multi-dose packages, preferably multi-dose packages.

Also, contrary to the expectations, it was found that notwithstanding the presence of high concentrations of high molecular weight silicon compound, the composition of the invention may be easily sprayed.

The composition of the invention may be applied by any possible method to the skin of a subject in need thereof, preferably by vaporisation (spraying).

The use of the composition as a spray, preferably by means of any known delivery system, allows an efficient application in all the external parts of the body of a subject, including parts which are difficult to treat with patches like for example foot and hand fingers and folder parts of the body like axilla or groin.

Therefore, the composition of the present invention used as a spray, can be easily applied also as self-medication and do not need to be spread. Also, the delivered solution quickly dries, without the formation of drops that normally occur when the spraying is performed with solutions comprising water or highly boiling solvents.

Any spray device can be used, for instance the Bag-On-Valve system (Type Aptar Pharma's Bag-On-Valve from Aptar Group-Italy), constituted by a valve welded to a bag and inserted in an aluminium container. The chamber pressurisation is obtained with an inert gas (usually nitrogen) in the same time of the valve crimping to the aluminium container. Then the bag is filled through the valve with the solutions obtainable as described in the examples of the present invention in the desired amount. Finally, the spray device is completed with an actuator and caps (type vapo spray D1, M1, H1 or S1 Aptar group—Italy).

A further advantage of the composition of the present invention as a spray is the treatment of pets, livestock and wild animals requiring an anti-inflammatory therapy. For example, the treatment by spray instead of gel or cream appears to be better deliverable to animals, such as racing horses suffering from leg contractures, or dogs that often suffer arthritic syndrome at hip, and more generally every animal requiring an anti-inflammatory therapy.

The composition of the invention may be administered once or more times per day, for instance 1 to 5 times/day.

The invention will now be illustrated by the following examples, in a non-limiting way.

### EXPERIMENTAL SECTION

### Definitions and Methods

DIEP diclofenac epolamine
HPLC High Performance Liquid Chromatography
h hour/hours

All densities were calculated at 25° C.

The quantitative assay applied in all the examples below is performed by a HPLC instrument with auto-injector, pump and UV detector, with a RP-18 column and with an isocratic mobile phase of 0.05 M phosphate buffer pH 7/acetonitrile/methanol 52/31/19 with a flux of 1 ml/min. With DIEP internal standard a known concentration solution with the mobile phase is prepared and different volumes are injected to obtain a calibration curve which to be used as quantitative reference curve must have a regression coefficient higher than 0.99. The resulting areas of the diclofenac signal measured at 284 nm are used to set-up the standard curve. The solution obtained in the examples of the present invention is diluted 100 times and 10 μl are analysed as described. The resulting area at 284 nm is compared with the calibration curve and the DIEP content is obtained considering the dilution factor and expressed as percentage of the declared content of diclofenac sodium equivalent.

### Comparative Example 1—1% Diclofenac Sodium Equivalent Solution without Dimethiconol 40, According to Italian Patent 1413980

5.5 litres of absolute ethanol (Fluka-CH) and 2.4 litres of isopropanol (Fluka-CH) are mixed in a 30 litre container keeping a mild stirring. To this mixture, 2 litres of isopropyl myristate (Fluka-CH) and 10 litres of Dowsil™ Q7-9180 Silicone Fluid 1.0 cP (from Dupont USA) are added, maintaining the mild stirring. To this solution 100 g. of menthol (Fluka-CH) are added and the stirring proceeds until complete dissolution. 216.18 g of DIEP (MW 411.3, 0.525 moles) obtained as described by Ziggiotti et al (EP-0271709) are added to the solution and the stirring is maintained until complete dissolution. All the operation is performed at 15-25° C. The solution obtained has a calculated density of 0.813 and a diclofenac sodium equivalent assay measured by quantitative HPLC analysis as described above of 109%.

### Example 1—1% Diclofenac Sodium Equivalent Solution with 5% Dimethiconol 40

260 g of absolute ethanol (Fluka-CH) and 110 g of isopropanol (Fluka-CH) are mixed in 1 litre container under mild stirring. To this mixture, 140 g of isopropyl myristate (Fluka-CH) and 423 g of Liveo™ Q7-9180 Silicone Fluid 0.65 cSt (from Dupont USA) and 50 g. of Liveo® ST-Dimethiconol 40 are added maintaining the mild stirring. To this solution 7 g. of Menthol (Fluka-CH) are added and the stirring proceeds until complete dissolution. 13.2 g of DIEP (MW 411.3, 0.032 moles) obtained as described by Ziggiotti et al (EP-0271709) are added to the solution and the stirring is maintained until complete dissolution. All the operations are performed at 15-25° C. The solution obtained has a calculated density of 0.814 and a diclofenac sodium equivalent assay measured by quantitative HPLC analysis as described above of 90%.

### Example 2—1% Diclofenac Sodium Equivalent Solution with 4% Dimethiconol 40

260 g of absolute ethanol (Fluka-CH) and 110 g of isopropanol (Fluka-CH) are mixed in 1 litre container under mild stirring. To this mixture, 140 g of isopropyl myristate (Fluka-CH) and 431 g of Liveo™ Q7-9180 Silicone Fluid 0.65 cSt (from Dupont USA) and 40 g. of Liveo® ST-Dimethiconol 40 are added maintaining the mild stirring. To this solution 7 g. of Menthol (Fluka-CH) are added and the stirring proceeds until complete dissolution. 13.2 g of DIEP (MW 411.3, 0.032 moles) obtained as described by Ziggiotti et al (EP-0271709) are added to the solution and the stirring is maintained until complete dissolution. All the operations are performed at 15-25° C. The solution obtained has a calculated density of 0.814 and a diclofenac sodium equivalent assay measured by quantitative HPLC analysis as described above of 100%.

### Example 3—1% Diclofenac Sodium Equivalent Solution with 6 Dimethiconol 40

260 g of absolute ethanol (Fluka-CH) and 110 g of isopropanol (Fluka-CH) are mixed in 1 litre container under mild stirring. To this mixture, 140 g of isopropyl myristate (Fluka-CH) and 413 g of Liveo™ Q7-9180 Silicone Fluid 0.65 cSt (from Dupont USA) and 60 g. of Liveo® ST-Dimethiconol 40 are added maintaining the mild stirring. To this solution 7 g. of Menthol (Fluka-CH) are added and the stirring proceeds until complete dissolution. 13.2 g of DIEP (MW 411.3, 0.032 moles) obtained as described by Ziggiotti et al (EP-0271709) are added to the solution and the stirring is maintained until complete dissolution. All the operations are performed at 15-25° C. The solution obtained has a calculated density of 0.814 and a diclofenac sodium equivalent assay measured by quantitative HPLC analysis as described above of 91%.

### Example 4—2% Diclofenac Sodium Equivalent Solution with 5% Dimethiconol 40

260 g of absolute ethanol (Fluka-CH) and 110 g of isopropanol (Fluka-CH) are mixed in 1 litre container under mild stirring. To this mixture, 140 g of isopropyl myristate (Fluka-CH) and 423 g of Liveo™ Q7-9180 Silicone Fluid 0.65 cSt (from Dupont USA) and 50 g. of Liveo® ST-Dimethiconol 40 are added maintaining the mild stirring. To this solution 7 g. of Menthol (Fluka-CH) are added and the stirring proceeds until complete dissolution. 26.4 g of DIEP (MW 411.3, 0.064 moles) obtained as described by Ziggiotti et al (EP-0271709) are added to the solution and the stirring is maintained until complete dissolution. All the operations are performed at 15-25° C. The solution obtained has a calculated density of 0.854 and a diclofenac sodium equivalent assay measured by quantitative HPLC analysis as described above of 95%.

### Example 5—3% Diclofenac Sodium Equivalent Solution with 5% Dimethiconol 40

256 g of absolute ethanol (Fluka-CH) and 159 g of isopropanol (Fluka-CH) are mixed in 1 litre container under mild stirring. To this mixture, 138 g of isopropyl myristate (Fluka-CH) and 366 g of Liveo™ Q7-9180 Silicone Fluid 0.65 cSt (from Dupont USA) and 49 g. of Liveo® ST-Dimethiconol 40 are added maintaining the mild stirring. To this solution 6.9 g. of Menthol (Fluka-CH) are added and the stirring proceeds until complete dissolution. 39.6 g of DIEP (MW 411.3, 0.096 moles) obtained as described by Ziggiotti et al (EP-0271709) are added to the solution and the stirring is maintained until complete dissolution. All the operations are performed at 15-25° C. The solution obtained has a calculated density of 0.854 and a diclofenac sodium equivalent assay measured by quantitative HPLC analysis as described above of 95%.

### Example 6—Permeation Test

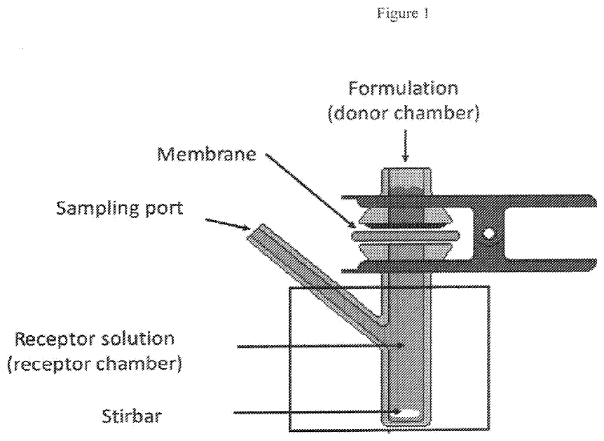

To test diclofenac delivery and skin permeation capability, the compositions as described in the Comparative Example 1 and Examples 1 to 3 have been compared in a Franz cell system. Permeation experiments were performed across isolated epidermis from pig ear skin, a well characterised model for human skin. Pig ears were obtained from a local slaughterhouse (Macello Annoni, Busseto, Italy) immediately after the death of the animal. Full thickness skin was excised from the outer region of the ear and separated from the underlying cartilage with a surgical blade. Hairs were trimmed carefully with scissors. Isolated epidermis was prepared, by soaking in water at 60° C. for 1 min and then peeling off with forceps, and frozen at −20° C. until use that occurred within 2 months. Franz-type vertical diffusion cells (Disa, Milano, Italy), with a diffusional area of 0.6 cm2, were used for permeation studies (FIG. 1).

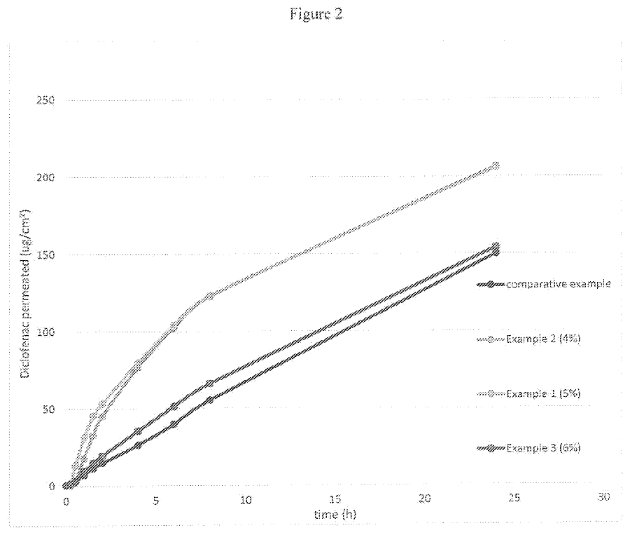

The isolated epidermis was mounted between the two halves of the cell, with the stratum corneum facing the donor compartment. The receptor compartment was filled with about 4 ml of PBS pH 7.4, magnetically stirred. The experiments had a duration of 24 hours and were conducted in thermostatic bath at 37° C. (32° C. on the skin surface). 60 μl of the solution to be tested (100 μl/cm2) were applied on the skin surface and at predetermined interval of time (0, 0.25, 0.5, 1, 1.5, 2, 4, 6, 8 and 24 h), 300 μl of solution were taken from the receptor compartment and replaced immediately with fresh solution. Samples were analysed by HPLC for the quantification of diclofenac as described above. Each experiment has been repeated 6 times. In FIG. 2 the diclofenac sodium equivalent cumulative concentration measured in the receiving compartment of the four products is reported up to 24 h.

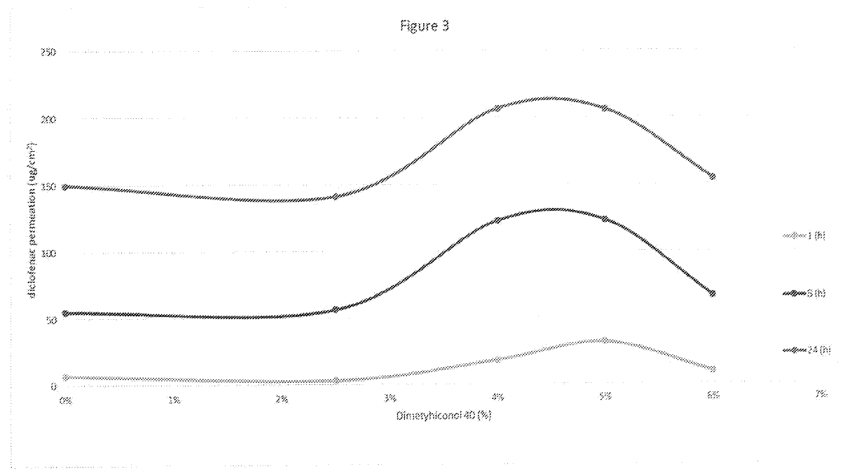

The data obtained show that the compositions of the Examples 1 to 3 have permeation properties in all time points higher than the reference product (Comparative Example 1). Among the testing compositions, composition of Example 1 shows the better permeation performance at all observation times. Interestingly the product of Example 3 shows less permeation than the other two products containing lower amounts of high molecular weight silicon compound. In FIG. 3 the permeation rate at 1, 8 and 24 hours in function of the high molecular weight silicon compound percentage are reported.

1. A topical pharmaceutical composition comprising:
(a) diclofenac or one of its pharmaceutically acceptable salts;
(b) at least one volatile silicon compound having a low dynamic viscosity;
(c) at least one high molecular weight silicon compound having a high dynamic viscosity;
(d) a solvent consisting of at least one saturated aliphatic alcohol having 2 to 4 carbon atoms, preferably a mixture of two saturated aliphatic alcohol having 2 to 4 carbon atoms;
(e) at least one permeation enhancer; and
(f) optionally a refreshing agent

2. The topical pharmaceutical composition according to claim 1, characterised in that it comprises:
(a) 0.1 to 3.5%, preferably 0.5 to 3.0% by weight (with respect to the total weight of the composition) of diclofenac or one of its pharmaceutically acceptable salts;
(b) 30 to 60%, preferably 35 to 55% by weight (with respect to the total weight of the composition) of a volatile silicon having a low dynamic viscosity;
(c) 3 to 6%, preferably 4 to less than 6% by weight (with respect to the total weight of the composition) of one high molecular weight silicon compound having a high dynamic viscosity;
(d) 20% to 50% by weight (with respect to the total weight of the composition) of a solvent consisting of a mixture at least one saturated aliphatic alcohol having 2 to 4 carbon atoms, preferably a mixture of two saturated aliphatic alcohol having 2 to 4 carbon atoms;
(e) 2% to 15%, preferably 6% to 15% by weight (with respect to the total weight of the composition) of at least one permeation enhancer, and
(f) 0 to 3%, preferably 0.1% to 3% in weight (with respect to the total weight of the composition) of a refreshing agent.

3. The topical pharmaceutical composition according to claim 1 or 2, characterised in that said volatile silicon having a low dynamic viscosity has a dynamic viscosity lower than or equal to 5 cP at 25° C. and that high molecular weight silicon compound having a high dynamic viscosity has a dynamic viscosity higher than or equal to 40 cP at 25° C.

4. The topical pharmaceutical composition according to any one of claims 1 to 3, characterised in that said pharmaceutically acceptable salts are diclofenac salts with an amine selected from methylamine, triethylamine, pyrrolidine, piperidine, morpholine, 1-ethylpiperidine, 2-aminoethanol, dimethylaminoethanol, diethylaminoethanol, epolamine and piperidineethanol, preferably epolamine or piperidineethanol.

5. The topical pharmaceutical composition according to any one of claims 1 to 4, characterised in that said volatile silicon compound having a low dynamic viscosity is selected from a (per)methylatedpolysiloxane having from 2 to 6 silicon atoms having a dynamic viscosity lower than 5 cP at 25° C., preferably selected from permethylated C2-C6-polysiloxanes, more preferably selected from hexamethyldisiloxane, octamethyltrisiloxane and decamethylpentasiloxane.

6. The topical pharmaceutical composition according to any one of claims 1 to 5, characterised in that said high molecular weight silicon compound having a high dynamic viscosity is selected from high molecular weight silicon compounds having a dynamic viscosity higher than 40 cP at 25° C., preferably a polydimethylsiloxane, more preferably Dimethiconol 40.

7. The topical pharmaceutical composition according to any one of claims 1 to 6, characterised in that said saturated aliphatic alcohol having 2 to 4 carbon atoms is selected from methanol, ethanol, n-propanol, iso-propanol, n-butanol, iso-butanol, sec-butanol and tert-butanol, preferably is a mixture of two saturated aliphatic alcohols having 2 and 3 carbon atoms.

8. The topical pharmaceutical composition according to claim 7, characterised in that said mixture is a mixture of ethanol and isopropanol.

9. The topical pharmaceutical composition according to any one of claims 1 to 8, characterised in that said skin permeation enhancer is selected from fatty alcohols, fatty acids, fatty acid esters, glycerol or saccharose monostearate, glycerol or saccharose monolinoleate, glycerol or saccharose monooleate; and fatty alcohol ethers from 10 to 20 carbon atoms, preferably isopropyl myristate.

10. The topical pharmaceutical composition according to any one of claims 1 to 9, characterised in that said refreshing agent is selected from menthol, camphor and mixture thereof.

11. The topical pharmaceutical composition according to any one of claims 1 to 10, characterised in that it comprises
(a) 0.1 to 3.5%, preferably 0.5 to 3.0% by weight (with respect to the total weight of the composition) of diclofenac or diclofenac epolamine salt or 1-(2-hydroxyethyl)piperidine salt;
(b) 30 to 60%, preferably 35 to 55% by weight (with respect to the total weight of the composition) of a volatile silicon selected from hexamethyldisiloxane, octamethyltrisioxane and decamethyleyclopentasiloxane;
(c) 3 to 6%, preferably 4 to less than 6% by weight (with respect to the total weight of the composition) of Dimethiconol 40;
(d) 20% to 50% by weight (with respect to the total weight of the composition) of a solvent consisting of a mixture of ethanol/isopropanol in a volume ratio of 1/0.4 to 1/3;
(e) 2% to 15%, preferably 6% to 15% by weight (with respect to the total weight of the composition) of isopropyl myristate; and
(f) 0 to 3%, preferably 0.1% to 3% in weight (with respect to the total weight of the composition) of menthol or camphor, or a mixture of the two.

12. The topical pharmaceutical composition according to any one of claims 1 to 11 characterised in that it has a density of from 0.6 to 1.0 g/mL, preferably from 0.7 to 0.9 g/mL at 25° C.

13. The topical pharmaceutical composition according to any one of claims 1 to 12 characterised in that it is administered by vaporisation.

14. The topical pharmaceutical composition according to any one of claims 1 to 13 for use in human and veterinary therapy.

15. The topical pharmaceutical composition according to claim 14 for use in the treatment of inflammation and pain.
